(19) United States
(12) Patent Application Publication
(10) Pub. No.: US 2004/0018218 A1
(43) Pub. Date: Jan. 29, 2004

(54) Composition comprising a ceramide precursor for improving natural or reconstructed epidermis, resulting skin equivalent

(76) Inventors: Michel Philippe, Wissous (FR); Isabelle Castiel, Jouy en Josas (FR); Corinne Ferraris, Paris (FR); Eric Arbey, Aulnay s/Bois (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/311,154

(22) PCT Filed: Jun. 11, 2001
(86) PCT No.: PCT/FR01/01800

(51) Int. Cl. A61K 7/48

(52) U.S. Cl. 424/401

(57) ABSTRACT

The invention concerns a composition comprising, in a physiologically acceptable medium, at least a ceramide precursor, the use of said compound in particular for reinforcing the lipid barrier of the epidermis and/or for improving the quality and properties of reconstructed epidermis, and a skin equivalent supplemented with said compound.

The present invention relates to the use of ceramide precursors in cosmetic or pharmaceutical compositions, in particular dermatological compositions, and to said compositions.

Ceramides, in their natural state, are the main components of the lipid layers of the epidermis. They are used in cosmetics, in their natural or synthetic form, in compositions intended, inter alia, to reduce drying of the skin or to confer better elasticity on the latter or alternatively intended for treatment of the hair.

Among ceramide precursors, sphingenine base, sphinganine base and 4-OH-sphinganine base (or phytosphingosine), which are essential links in the ceramide structures present in the animal and plant world, have been known for a long time.

Sphingenine in particular has been claimed as active principle making it possible to improve damaged skin (JP62120308) and to replace skin cells (WO 94/23694).

Furthermore, it was discovered not very long ago that 6-OH-sphingenine base is present in certain ceramide structures extracted from human stratum corneum. This is described in particular in ‘Journal of Lipid Research, 35, 2060-2068, 1994’.

However, it is known that sphingenine base exhibits in particular the disadvantage of clearly being involved in apoptosis phenomena, as was described in ‘Cell Death Differ. 1(2), 93-99, 1994’.

Furthermore, when this base is incorporated into a ceramide, it can lead to a decrease in cell viability; this is disclosed in particular in EP790053.

An aim of the present invention is to provide the use, in particular in cosmetics and in pharmaceuticals, of a novel base which does not exhibit the disadvantages of the prior art, which has in particular very low toxicity and which, furthermore, is predominantly present in the skin.

This novel base can be employed in a very specific way for caring for the skin.

In particular, the compounds disclosed in the present invention can make it possible to strengthen the lipid barrier of the epidermis and/or to re-establish or maintain the integrity of the stratum corneum.

They can therefore be employed for improving the surface appearance and the moisturizing of the skin and for protecting it, in particular protecting dry and rough skin.

They can also be used as essential nutritional agents for keratinous substances (skin, hair, eyelash, nail).

Furthermore, the compositions comprising these compounds can advantageously be employed for improving and/or maintaining the lipid content of the human epidermis, in vivo and in vitro, and thus improving the quality and the properties, such as lipid content and/or barrier property, of reconstructed epidermes and/or epidermal cell cultures.

A subject matter of the present invention is a composition comprising, in a physiologically acceptable medium, at least one compound of formula (I):

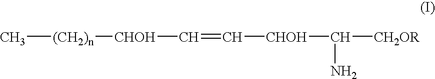

in which

n is an integer between 3 and 17 inclusive, and

R represents a hydrogen atom, a sulfate radical —O—SO₃ or a phosphate radical —O—PO₃,

and its salts.

Another subject matter of the invention is the use of at least one compound of formula (I) or of a composition comprising it, for:

strengthening the lipid barrier of the epidermis, in particular of dry and/or rough and/or damaged skin, and/or

re-establishing or maintaining the integrity of the stratum corneum, and/or

improving the surface appearance and/or the moisturizing of the skin, and/or

for protects the skin, in particular dry and rough skin, and/or

as essential nutritional agent for keratinous substances (skin, hair, eyelash, nail), and/or

for strengthening the lipid barrier of reconstructed skin, or skin equivalent, and/or

for improving and/or maintaining the lipid content of the human epidermis, in vivo and in vitro, and/or

for improving the quality and the properties, such as lipid content and/or barrier property, of reconstructed epidermes and/or epidermal cell cultures.

Another subject matter of the invention is a skin equivalent supplemented with at least one compound of formula (I).

The composition according to the invention thus comprising at least one compound of formula (I):

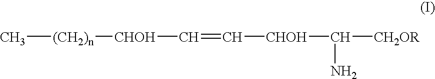

in which:

n is an integer between 3 and 17 inclusive, and

R represents a hydrogen atom, a sulfate radical —O—SO₃ or a phosphate radical —O—PO₃,

and its salts.

Preferably, n is between 8 and 14 and even better still between 10 and 12. Preferentially, n=11.

Preferably, R represents a hydrogen atom.

The salts of the compounds of formula (I) can be organic, for example salts of C₂₋₃₀ carboxylic acids or of C₃₋₃₀ hydroxy acids, in particular of C₃₋₃₀ α-hydroxy acids.

These salts may also be inorganic, for example in the form of sulfates or of halides.

Of course, the compounds of formula (I) can be provided in the form of a mixture of isomers or in the form of a pure isomer.

A mixture of different compounds of formula (I) can be envisioned.

The compounds of formula (I) can be prepared according to described synthetic processes well known to a person skilled in the art, for example as described or inspired by “Mendeleev Commun., 108-110, 1992” or “Tetrahedron Letters, 34, No. 7, 1191-1194, 1993”.

The composition according to the invention additionally comprises a physiologically acceptable medium, that is to say, when an application is envisioned in the cosmetic or pharmaceutical fields, a cosmetically or pharmaceutically acceptable medium, thus compatible with application to keratinous substances, such as the skin, nails, hair, eyelashes and eyebrows, mucous membranes and semimucous membranes, and any other cutaneous region of the body and face.

In this case, the composition according to the invention can comprise the compounds of formula (I) in an amount of 0.005% to 20% by weight, preferably of 0.01 to 10% by weight, in particular 0.05 to 5%, indeed even 0.1 to 1%, by weight, with respect to the total weight of the composition.

The physiologically acceptable medium in which the compounds according to the invention can be employed, and its constituents, their amounts, the pharmaceutical dosage form of the composition and its method of preparation, can be chosen by a person skilled in the art on the basis of his general knowledge according to the type of composition desired.

Generally, this medium can be anhydrous or aqueous. It can thus comprise an aqueous phase and/or a fatty phase.

The composition can thus comprise an aqueous phase which can comprise water, a floral water, such as cornflower water, a mineral water, such as water from Vittel, water from Lucas or water from La Roche-Posay, and/or a thermal water.

Said aqueous phase can additionally comprise alcohols, such as C₁-C₆ monoalcohols, among which may be mentioned ethanol, propanol, butanol, isopropanol or isobutanol; and/or polyols, such as glycerol, butylene glycol, isoprene glycol, propylene glycol or polyethylene glycol.

The composition according to the invention can also comprise a fatty phase composed in particular of fatty substances which are liquid at 25° C., such as volatile and nonvolatile oils of animal, vegetable, mineral or synthetic origin; of fatty substances which are solid at 25° C., such as waxes of animal, vegetable, mineral or synthetic origin; of pasty fatty substances; of gums; or of their mixtures.

The volatile oils are generally oils having a saturated vapour pressure at 25° C. at least equal to 0.5 millibar (i.e. 50 Pa).

Mention may be made, among the constituents of the fatty phase, of:

cyclic volatile silicones having from 3 to 8 silicon atoms and preferably from 4 to 6. They are, for example, cyclotetradimethylsiloxane, cyclopenta-dimethylsiloxane or cyclohexadimethylsiloxane;

cyclocopolymers of the dimethylsiloxane/methylalkyl-siloxane type;

linear volatile silicones having from 2 to 9 silicon atoms. They are, for example, hexamethyldisiloxane or a PDMS of low viscosity (1 cSt). Mention may also be made of hexamethyldisiloxane and alkyltrisiloxanes, such as hexylheptamethyltrisiloxane or octylheptamethyl-trisiloxane;

volatile hydrocarbon-based oils, such as isoparaffins and in particular isododecane;

poly(C₁-C₂₀)alkylsiloxanes and in particular those with end trimethylsilyl groups, among which may be mentioned linear polydimethylsiloxanes and also alkylmethylpolysiloxanes, such as cetyl dimethicone (CTFA name);

silicones modified by optionally fluorinated aliphatic and/or aromatic groups or by functional groups, such as hydroxyl, thiol and/or amine groups;

phenylated silicone oils;

oils of animal, vegetable or mineral origin and in particular animal or vegetable oils formed by esters of fatty acid and of polyols, in particular liquid triglycerides, for example sunflower, maize, soybean, cucumber, grape seed, sesame, hazelnut, apricot, almond or avocado oils; fish oils, capric/caprylic triglyceride, or vegetable or animal oils of formula R₁COOR₂ in which R₁ represents the residue of a higher fatty acid comprising from 7 to 19 carbon atoms and R₂ represents a branched hydrocarbon-based chain comprising from 3 to 20 carbon atoms, for example Purcellin oil; liquid paraffin, liquid petrolatum, perhydrosqualene, or wheat germ, calophyllum, sesame, macadamia, grape seed, rapeseed, coconut, groundnut, palm, castor, jojoba, olive or cereal germ oils; fatty acid esters; alcohols; acetylglycerides; octanoates, decanoates or ricinoleates of alcohols or of polyalcohols; fatty acid triglycerides; or glycerides;

fluorinated and perfluorinated oils;

silicone gums;

waxes of animal, vegetable, mineral or synthetic origin, such as microcrystalline waxes, paraffin wax, petrolatum wax, ozokerite or montan wax; beeswax or lanolin and its derivatives; candelilla, ouricury, carnauba or japan waxes, cocoa butter or cork fiber or sugar cane waxes; hydrogenated oils which are solid at 25° C., ozokerites, or fatty esters and glycerides which are solid at 25° C.; polyethylene waxes and waxes obtained by the Fischer-Tropsch synthesis; hydrogenated oils which are solid at 25° C.; lanolins, fatty esters which are solid at 25° C.; silicone waxes; or fluorinated waxes.

When the composition according to the invention is provided in the form of an emulsion, it can in addition optionally comprise a surfactant, preferably in an amount of 0.01 to 30% by weight with respect to the total weight of the composition. The composition according to the invention can also comprise at least one coemulsifier which can be chosen from oxyethylenated sorbitan monostearate, fatty alcohols, such as stearyl alcohol or cetyl alcohol, or esters of fatty acids and of polyols, such as glyceryl stearate.

In addition, the composition according to the invention can comprise a particulate phase which can comprise pigments and/or pearlescent agents and/or fillers commonly used in cosmetic compositions.

The pigments can be present in a proportion of 0-20% by weight with respect to the total weight of the composition and preferably in a proportion of 2-15%. It can be white or colored, inorganic and/or organic, and have conventional or nanometric size. Mention may be made, among inorganic pigments and nanopigments, of titanium, zirconium or cerium dioxides, and also zinc, iron or chromium oxides, titanium dioxide nanopigments or ferric blue. Mention may be made, among organic pigments, of carbon black, and lakes commonly employed to confer a make-up effect on the lips and skin, which lakes are calcium, barium, aluminum or zirconium salts of acid dyes, such as haloacid, azo or anthraquinone dyes.

The pearlescent agents can be present in the composition in a proportion of 0-20% by weight, preferably at a high level of the order of 2-15% by weight. Mention may be made, among the pearlescent agents which can be envisioned, of natural mother-of-pearl, mica covered with titanium oxide, with iron oxide, with natural pigment or with bismuth oxychloride, and colored titanium oxide-coated mica.

The fillers, which can be present in the composition in a proportion of 0-20% by weight with respect to the total weight of the composition, preferably 2-10%, can be inorganic or synthetic and lamellar or nonlamellar. Mention may be made of talc, mica, silica, kaolin, nylon and polyethylene powders, Teflon, starch, boron nitride, microspheres, such as Expancel (Nobel Industry), polytrap (Dow Corning) and silicone resin microbeads (Tospearls from Toshiba, for example).

The composition can also comprise water-soluble dyes, chosen from dyes conventional in the field under consideration, such as the disodium salt of ponceau, the disodium salt of alizarin green, quinoline yellow, the trisodium salt of amaranth, the disodium salt of tartrazine, the monosodium salt of rhodamine, the disodium salt of fuchsin, or xanthophyll.

In a known way, the composition according to the invention can comprise the adjuvants usual in the field under consideration, such as hydrophilic or lipophilic gelling agents, hydrophilic or lipophilic additives, hydrophilic or lipophilic, in particular cosmetic or pharmaceutical, active principles, preservatives, antioxidants, solvents, fragrances, fillers, pigments, pearlescent agents, UV screening agents, odor absorbers and dyes. The nature and the amount of these adjuvants can be chosen by a person skilled in the art on the basis of his general knowledge so as to obtain the presentation form desired for the composition. In any event, a person skilled in the art will take care to choose all the optional additional compounds and/or their amounts so that the advantageous properties of the composition according to the invention are not, or not substantially, detrimentally affected by the envisioned addition.

In a preferred embodiment of the invention, the compositions comprising said compounds of formula (I) can additionally comprise compounds conventionally employed in the field of application under consideration, such as active principles which can help in preventing or in improving keratinous substances, in particular the skin and even better still dry, rough and/or damaged skin. Mention may in particular be made of the other ceramide bases, such as sphingenine, sphinganine or 4-OH-sphinganine; ceramides; glycoceramides; C₁₄₋₃₀ fatty acids; triglycerides; sterols, such as cholesterol; or fatty substances commonly used in cosmetics.

The compounds according to the invention find a specific application in compositions intended for strengthening the lipid barrier of the skin, in particular in compositions intended to care for the skin, in particular dry and/or rough and/or damaged skin.

For application to the skin, the composition can have the form in particular of an aqueous or oily solution; of a dispersion of the lotion or serum type; of emulsions with a liquid or semiliquid consistency of the milk type obtained by dispersion of a fatty phase in an aqueous phase (O/W) or vice versa (W/O); of suspensions or emulsions with a soft consistency of the aqueous or anhydrous gel or cream type; of microcapsules or microparticles; or of vesicular dispersions of ionic and/or nonionic type.

For application to the hair, the composition can be in the form of aqueous, alcoholic or aqueous/alcoholic solutions; in the form of creams, gels, emulsions or mousses; or in the form of aerosol compositions also comprising a pressurized propellant.

The composition according to the invention can be provided in particular in the form:

of a product for caring for, treating or protecting keratinous substances and in particular the skin of the face or body, including the scalp, such as a (day, night or moisturizing) care composition for the face or body; an antiwrinkle or antiage composition for the face; a mattifying composition for the face; a composition for irritated skin; a make-up-removing composition; or an optionally aftersun, in particular moisturizing, body milk;

of a sun protection, artificial tanning (self-tanning) or aftersun care composition;

of a hair composition and in particular a cream or a gel for protecting from the sun; a composition for caring for the scalp, in particular a composition for combating hair loss or for promoting hair regrowth; or an antiparasitic shampoo;

of a product for making up keratinous substances, such as a foundation, a tinted cream, a blusher, an eyeshadow, a free or compact powder, a concealer stick, a cover stick, a lipstick or a lipcare product; or a nail varnish, a nail care product, a mascara, a treating mascara or an eyeliner.

The compositions according to the invention find a preferred application as a composition of moisturizing type for caring for the skin of the face and as sun protection or aftersun composition.

The compositions according to the invention find a very particular application in caring for keratinous substances, in particular the skin, nails, eyelashes or hair, and especially dry and/or rough and/or damaged skin.

The compounds according to the invention also find a particular application in strengthening the lipid barrier of reconstructed skin, also known as skin equivalent.

This is because it has been found that supplementing said reconstructed skin with compounds according to the invention makes it possible to improve the appearance and the barrier properties of this skin and to render it structurally closer to normal skin. A novel skin equivalent, or novel reconstructed skin, supplemented with at least one compound of formula (I), in particular with 6-OH-sphingenine, can thus be obtained.

It is known that it has been possible to develop models reasonably close to human skin. Mention may be made, for example, of the models disclosed in patent applications EP-A-285 471, EP-A-285 474, EP-A-789 074, EP-A-502 172, EP-A-418 035, WO-A-9116010, EP-A-197 090, EP-A-20 753, FR-A-2 665 175 and FR-A-2 689 904 and in the as yet unpublished application 99FR-04 970 on behalf of the Applicant Company.

Very generally, the models of reconstructed skin disclosed in these documents comprise human keratinocytes in combination or not in combination with other skin cells, such as melanocytes and/or Langerhans cells, deposited on a support, often a dermis equivalent, and cultured under conditions such that they enter a program of differentiation resulting in the formation of an epidermis equivalent. The dermis equivalents described to date are either artificial membranes, such as, for example, Millipore® filters, subcutaneous substitutes based on collagen, plastic or any other support compatible with cell viability, or supports which are more complex in order to render them closer to the natural dermis, such as prede-epidermized dermis or collagen/fibroblast mixed lattices. In collagen/fibroblast mixed lattices, the combination of native collagen and of isolated human fibroblasts results in the preparation of a dermis equivalent which mimics a dermis which has not been subjected to the action of time.

However, it has been found that none of the abovementioned skin equivalents comprising compounds of formula (I) and in particular 6-OH-sphingenine.

The use of the compounds of formula (I) as defined above thus exhibits the great advantage of making available to researchers a novel skin equivalent supplemented with at least one compound of formula (I) and more particularly with 6-OH-sphingenine.

In particular, said skin equivalent can be treated with a composition comprising, in an aqueous medium, an amount of 10⁻⁷ to 1% by weight of compound of formula (I), in particular of 5.10⁻⁵ to 5.10⁻²% by weight and especially of 5.10⁻³ to 10⁻²% by weight, with respect to the total weight of the composition.

A composition is thus obtained which is provided in the form of a composition for the treatment of reconstructed skin or skin equivalents and epidermal cell cultures.

The invention is illustrated in more detail in the following examples.

### EXAMPLE 1

Moisturizing Oil-in-Water Emulsion

An O/W emulsion is prepared comprising:

| | | |
|---|---|---|
| Maize germ oil | 2 | g |
| Glyceryl monostearate | 3 | g |
| Polyethylene glycol 400 | 3 | g |
| Gelling agent (Carbopol 941 ®, | 0.2 | g |
| sold by Goodrich) | | |
| Isopropyl myristate | 3.0 | g |
| 6-OH-Sphingenine (compound of | | |
| formula (I) with n = 11 and R = H) | 0.1 | g |
| Cetyl alcohol | 3 | g |
| Stearyl alcohol | 3 | g |
| NaOH | 0.008 | g |
| Propylene glycol | 5.0 | g |

| | |
|---|---|
| Preservatives | q.s. |
| Water | q.s. for 100 g |

### EXAMPLE 2

Moisturizing Water-in-Oil Emulsion

A W/O emulsion is prepared comprising:

| | | |
|---|---|---|
| Liquid petrolatum | 10 | g |
| Mixture of fatty substances | 20 | g |
| (Protegin X from Goldschmidt) | | |
| Sunflower oil | 15 | g |
| 6-OH-Sphingenine | 0.05 | g |
| Magnesium sulfate | 0.5 | g |
| Glyceryl | 5 | g |
| Glyceryl cocoate 7EO | 4 | g |

| | |
|---|---|
| Fragrance | q.s. |
| Preservatives | q.s. |
| Water | q.s. for 100 g |

### EXAMPLE 3

Aqueous Gel

An aqueous gel is prepared comprising:

| | | |
|---|---|---|
| Gelling agent (Carbopol 941 ®, | 0.2 | g |
| sold by Goodrich) | | |
| Diethylene glycol monoethyl ether | 5 | g |
| Triethanolamine | 0.3 | g |
| Propylene glycol | 3 | g |
| NaOH | 0.007 | g |
| 6-OH-Sphingenine | 0.1 | g |

| | |
|---|---|
| Preservatives | q. s. |
| Water | q.s. for 100 g |

### EXAMPLE 4

Liposome-Based Cream

A cream with nonionic liposomes is prepared comprising:

| | | |
|---|---|---|
| Gelling agent (Carbopol 941 ®, | 0.2 | g |
| sold by Goodrich) | | |
| Diethylene glycol monoethyl ether | 3 | g |
| Triethanolamine | 0.2 | g |
| Polyglyceryl-3 cetyl ether | 3.8 | g |
| Coemulsifier (β-sitosterol) | 3.8 | g |
| Dicetyl phosphate | 0.4 | g |
| NaOH | 0.007 | g |
| 6-OH-Sphingenine | 0.15 | g |
| Sunflower oil | 35 | g |

| | |
|---|---|
| Fragrance | q.s. |
| Preservatives | q.s. |
| Water | g.s. for 100 g |

### EXAMPLE 5

A simplex composition is prepared and is applied to a reconstructed skin.

A PBS (phosphate buffer saline) saline solution is prepared comprising:

| | |
|---|---|
| KH₂PO₄ | 0.21 g/l |
| NaCl | 9.00 g/l |
| Na₂H₃PO₄.7H₂O | 0.726 g/l |
| 6-OH-Sphingenine | 0.01 g/l |

1. A composition comprising, in a physiologically acceptable medium, at least one compound of formula (I): 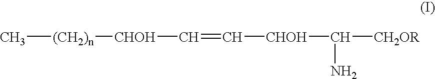
in which
n is an integer between 3 and 17 inclusive, and
r represents a hydrogen atom, a sulfate radical —O—SO₃ or a phosphate radical —O—PO₃, and its salts.

2. The composition as claimed in claim 1, in which n is between 8 and 14, in particular between 10 and 12, and preferentially n=11.

3. The composition as claimed in either of the preceding claims, in which R represents a hydrogen atom.

4. The composition as claimed in one of the Preceding claims, in which the salts of the compounds of formula (I) are organic, for example salts of C₂₋₃₀ carboxylic acids or of C₃₋₃₀ hydroxy acids, in particular of C₃₋₃₀ α-hydroxy acids, or are inorganic, for example in the form of sulfates or of halides.

5. The composition as claimed in one of the preceding claims, in which the physiologically acceptable medium is a cosmetically or pharmaceutically acceptable medium and in which the compound of formula (I) is present in an amount of 0.005% to 20% by weight, preferably of 0.01 to 10% by weight, in particular 0.05 to 5%, indeed even 0.1 to 1%, by weight, with respect to the total weight of the composition.

6. The composition as claimed in one of the preceding claims, furthermore comprising active principles which can help in preventing or in improving keratinous substances, in particular the skin and even better still dry, rough and/or damaged skin, such as sphingenine, sphinganine or 4-OH-sphinganine; ceramides; glycoceramides; C₁₄₋₃₀ fatty acids; triglycerides; sterols, such as cholesterol; or fatty substances commonly used in cosmetics.

7. The composition as claimed in one of the preceding claims, which is provided in the form:
of a product for caring for, treating or protecting keratinous substances and in particular the skin of the face or body, including the scalp, such as a (day, night or moisturizing) care composition for the face or body; an antiwrinkle or antiage composition for the face; a mattifying composition for the face; a composition for irritated skin; a make-up-removing composition; or an optionally aftersun, in particular moisturizing, body milk;
of a sun protection, artificial tanning (self-tanning) or aftersun care composition;
of a hair composition and in particular a cream or a gel for protecting from the sun; a composition for caring for the scalp, in particular a composition for combating hair loss or for promoting hair regrowth; or an antiparasitic shampoo;
of a product for making up keratinous substances, such as a foundation, a tinted cream, a blusher, an eyeshadow, a free or compact powder, a concealer stick, a cover stick, a lipstick or a lipcare product; or a nail varnish, a nail care product, a mascara, a treating mascara or an eyeliner.

8. The composition as claimed in one of the preceding claims, which is provided in the form of a composition for caring for keratinous substances, in particular the skin, nails, eyelashes or hair, and especially dry and/or rough and/or damaged skin.

9. The composition as claimed in one of claims 1 to 4, comprising, in an aqueous medium, an amount of 10⁻⁷ to 1% by weight of compound of formula (I), in particular of 5.10⁻⁵ to 5.10⁻²% by weight and especially of 5.10⁻³ to 10⁻²% by weight, with respect to the total weight of the composition.

10. The composition as claimed in claim 9, which is provided in the form of a composition for the treatment of reconstructed skin or skin equivalents and epidermal cell cultures.

11. The use of at least one compound of formula (I): 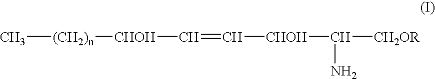
in which:
n is an integer between 3 and 17 inclusive, and
R represents a hydrogen atom, a sulfate radical —O—SO₃ or a phosphate radical —O—P)O₃,
and its salts,
or of a composition comprising it, for:
strengthening the lipid barrier of the epidermis, in particular of dry and/or rough and/or damaged skin, and/or
re-establishing or maintaining the integrity of the stratum corneum, and/or
improving the surface appearance and/or the moisturizing of the skin, and/or
for protects the skin, in particular dry and rough skin, and/or
as essential nutritional agent for keratinous substances (skin, hair, eyelash, nail), and/or
for strengthening the lipid barrier of reconstructed skin, or skin equivalent, and/or
for improving and/or maintaining the lipid content of the human epidermis, in vivo and in vitro, and/or
for improving the quality and the properties, such as lipid content and/or barrier property, of reconstructed epidermes and/or epidermal cell cultures.

12. A skin equivalent supplemented with at least one compound of formula (I): 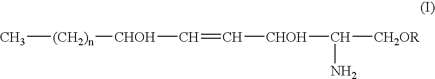
in which:
n is an integer between 3 and 17 inclusive, and
R represents a hydrogen atom, a sulfate radical —O—SO₃ or a phosphate radical —O—PO₃,
and its salts.
